(19) United States
(12) Patent Application Publication
(10) Pub. No.: US 2022/0370312 A1
(43) Pub. Date: Nov. 24, 2022

(54) Method of Combining Pigmentation Agents in Hair Dyeing and Related Products for Generating Cool Tones

(71) Applicants: Ayman Eldabli, Country Club Hills, IL (US); Mohamed Eldabli, FLOSSMOOR, IL (US); Lea Eldabli, COUNTRY CLUB HILLS, IL (US); Daniel Tetreault, Feeding Hills, MA (US)

(72) Inventors: Ayman Eldabli, Country Club Hills, IL (US); Mohamed Eldabli, FLOSSMOOR, IL (US); Lea Eldabli, COUNTRY CLUB HILLS, IL (US); Daniel Tetreault, Feeding Hills, MA (US)

(21) Appl. No.: 17/697,362

(22) Filed: Mar. 17, 2022

Related U.S. Application Data
(60) Provisional application No. 63/191,498, filed on May 21, 2021.

(51) Int. Cl. A61K 8/22; A61Q 5/10; A61Q 5/06; A61Q 5/12; A61K 8/64

(52) U.S. Cl. CPC A61K 8/22; A61Q 5/10; A61Q 5/06; A61Q 5/12; A61K 8/645; A61K 2800/882; A61K 2800/884; A61K 2800/43

(57) ABSTRACT

A method to produce an effective cool tone with blonde hair color, while performing the hair dyeing process. To that end, the desired blonde hair color may be achieved by incorporating pigmented capsules and/or any pigmentation agent into the hair bleach mixture and/or the hair peroxide mixture. The pigmentation agent provides a cool tone effect while simultaneously removing unwanted yellow and orange undertones from hair during the hair bleaching process. Accordingly, the method includes producing pigmented hair bleach mixture and pigmented hair peroxide mixtures for performing the hair lightening process. However, it is important to note the scope of the method includes incorporation of pigmented capsules and/or any pigmentation agent into any and all related hair dyeing or hair coloring and hair care products to achieve toning of hair.

### FIELD OF THE INVENTION

The present invention generally relates to hair lighteners and developers. More specifically, the incorporation of pigmented capsules and/or any pigmentation agent to hair bleach, hair peroxide mixtures and/or other related hair dyeing or hair coloring products.

### BACKGROUND OF THE INVENTION

Typically, when dealing with hair lighteners or related products for a blonde color outcome, there are two main hair mixtures involved. A hair bleach mixture is utilized to lighten the hair, meanwhile a hair peroxide mixture is utilized to develop the desired blonde hair color. Unfortunately, when a user's hair is dark, the aforementioned procedure presents some challenges. The bleach and peroxide mixture usually result in orange and/or yellow brassy undertones which are not typically the hair tone the user wants. Typical solutions to this usually involve long and tedious processes which involve various progressive lightening sessions. Therefore, an effective method or a product through which a user may achieve a blonde hair color without compromising the hair color integrity, is a rare find in the current market.

An objective of the present invention is to provide a unique and effective solution to the above-mentioned problems, which allows for hair toning during the hair dyeing or hair coloring process. According to the preferred embodiment, the desired result for blonde hair may be achieved by incorporating pigmented capsules and/or any pigmentation agent into the hair bleach mixture and/or the hair peroxide mixture. However, it is important to note the scope of the present invention includes incorporation of pigmented capsules and/or any pigmented agent into any and all related hair dyeing or hair coloring products and is not limited to hair bleach mixture and hair peroxide mixture.

The following document aims to provide an accurate and detailed description of the present invention without limiting the scope of the invention, and the accompanying figures are only intended to help illustrate the present invention. Thus, the accompanying figures do not limit the scope of the invention in any way, shape or form.

### DETAIL DRAWINGS OF THE INVENTION

FIG. 1 is a flow diagram describing the overall process followed by a method of the present invention.

FIG. 2 is a flow diagram of an extended process of the method, wherein combining a pigmentation agent with a plurality of hair care products is described.

FIG. 3 is a flow diagram describing a sub process of the method of the present invention, wherein pigmented capsules are used as the pigmentation agent.

FIG. 4 is a schematic overview, wherein a pigmentation agent is combined with a hair peroxide mixture.

FIG. 5 is a schematic overview, wherein pigmented capsules are combined with a hair bleach mixture.

FIG. 6 is a block diagram illustrating possible mixtures that are compatible with the pigmented capsules.

FIG. 7 is a block diagram illustrating combination of the pigmentation agent in both the lightener and the developer, as well as the combination of the lightener and the developer.

### DETAIL DESCRIPTIONS OF THE INVENTION

All illustrations of the drawings are for the purpose of describing selected versions of the present invention and are not intended to limit the scope of the present invention.

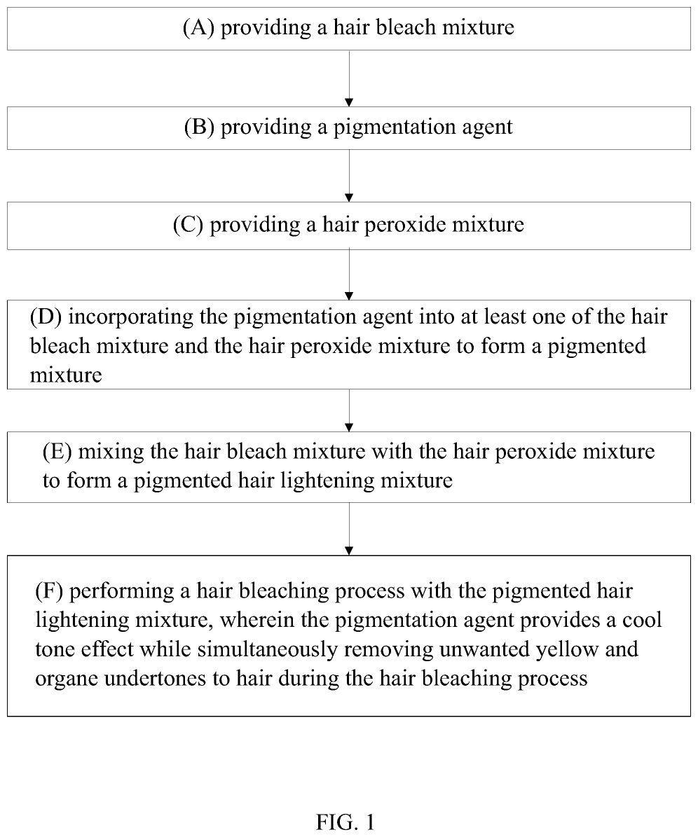

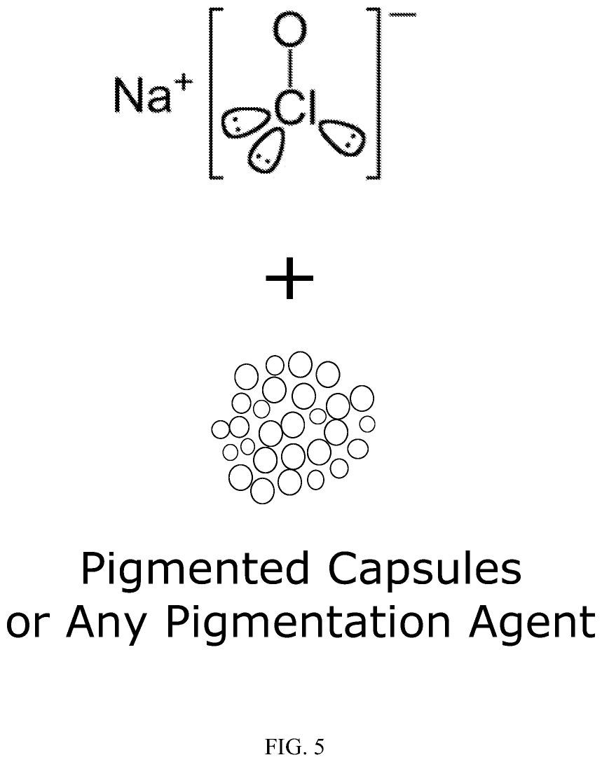

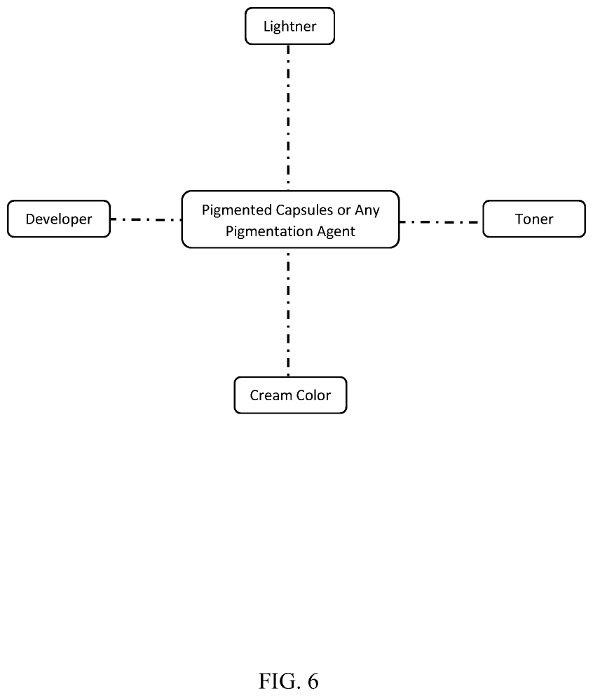

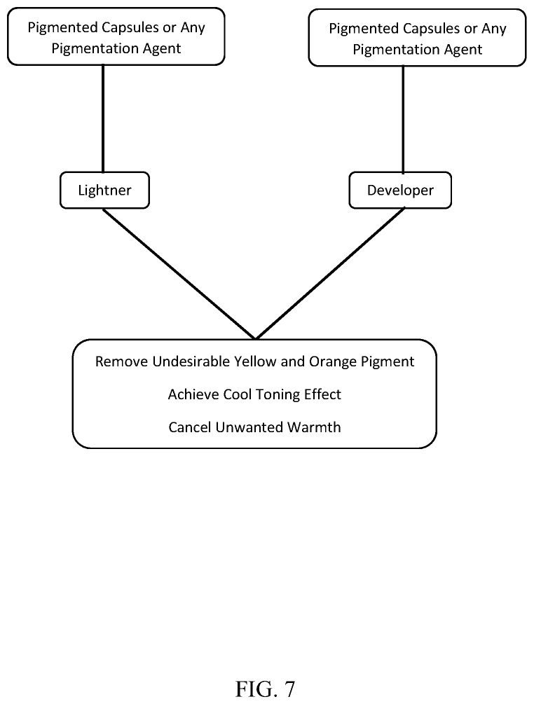

In reference to FIG. 1 through FIG. 7, the present invention is a method of combining pigmented capsules and other pigmentation agents for hair dyeing or hair coloring related products. It is an aim of the present invention to provide a unique and effective solution which allows for hair toning during the hair dyeing or hair coloring process. According to the preferred embodiment, the desired blonde hair color may be achieved by incorporating pigmented capsules and/or any pigmentation agent into the hair bleach mixture and/or the hair peroxide mixture. However, it is important to note the scope of the present invention includes incorporation of pigmented capsules and/or any pigmented agent into any and all related hair dyeing products and is not limited to hair bleach mixture and hair peroxide mixture.

The following description is in reference to FIG. 1 through FIG. 7. According to a preferred embodiment, the present invention is a method for providing desired color tone in hair dyeing or hair coloring process. To accomplish this, the overall process of the method begins by providing a hair bleach mixture (step A), providing a pigmentation agent (step B) and providing a hair peroxide mixture (step C). In the preferred embodiment, the present invention focuses on hair bleach and hair peroxide mixtures, however the present invention is not limited to such configurations. Preferably, the hair bleach mixture is often referred to as lightener in the hair styling and coloring industry, and the hair peroxide mixture is typically referred to as developer in the hair styling and coloring industry. Further, the pigmentation agent may comprise any and all pigmentation agents which fulfill production, user and/or chemical requirements. Examples of such pigmentation agents include, but are not limited to liquid pigmentation agents, granular pigmentation agents and/or any feasible pigmentation agent.

According to the preferred embodiment, the overall method subsequently follows by incorporating the pigmentation agent into at least one of the hair bleach mixture, and the hair peroxide mixture to form a pigmented mixture (step D). More specifically, the pigmented mixture may be a pigmented hair bleach mixture and/or a pigmented hair peroxide mixture. Incorporating pigmented capsules and/or any type of pigmentation agent into hair bleach mixture results in a mixture which allows for toning of hair during the bleaching process. Similarly, the presence any type of pigmentation agent in the developer mixture or hair peroxide mixture aids in cancelling out unwanted warmth which may occur during the lightening process. Accordingly, the hair bleach mixture is mixed with the hair peroxide mixture, to form a pigmented hair lightening mixture (step E). Typically, the combination of the hair bleach mixture and the hair peroxide mixture is used to bleach/lighten dark hair. Subsequently, the method continues by performing a hair bleaching process with the pigmented hair lightening mixture, wherein the pigmentation agent provides a cool tone effect while simultaneously removing unwanted yellow and orange undertones from hair during the hair bleaching process (step F). The amount of pigmentation agent being mixed with the hair bleach mixture and the hair peroxide mixture may be customized according to the color requirement. For example, the more the pigmentation agent, lesser warmth or more cool tone may be achieved in the resulting hair color. Additionally, the pigmented hair bleaching mixture provides protection while providing simultaneous cosmetic treatment of hydration and emollience while enhancing the effectiveness of the coloring and bleaching procedure. Thus, the pigmentation agent helps with providing the right blonde hair color with the correct cool tones. For the preferred embodiment the hair peroxide mixture or developer comprises vegetable collagen for improving hair strength and flexibility, but it is not limited to such. That is, the hair peroxide mixture may comprise any and all ingredients which provide the same benefits.

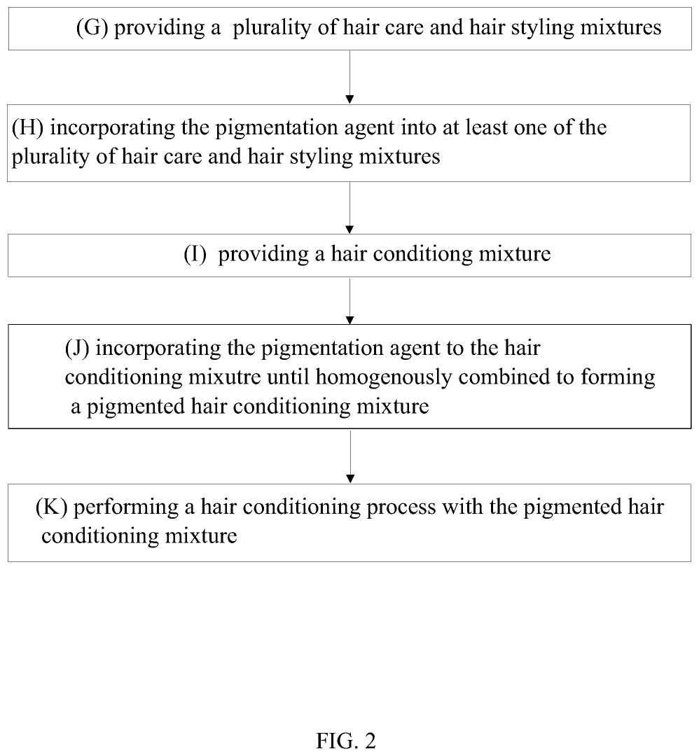

In reference to FIG. 2, the pigmentation agent may also be incorporated into other similar hair care mixture and/or solutions which are involved in the process of hair toning and/or lightening to maintain and provide the desired cool toned color effect. To that end, an extended process of the method begins by providing a plurality of hair care and hair styling mixtures (step G). The plurality of hair care and hair styling mixtures may comprise any products that are known to one of ordinary skill in the art, as long as the intents of the present invention are not altered. Examples of such products includes, but are not limited to shampoos, leave-in conditioners, serums, other color treatment products etc. The extended process continues by incorporating the pigmentation agent to at least one of the plurality of hair care and hair styling mixtures (step H). In other words, the pigmentation agent may be incorporated to one or more of the plurality of hair care and hair styling mixtures, such that the desired cool toned blonde hair is retained and not lost while using any of the plurality of hair care and hair styling mixtures. Similarly, the method may be extended to providing a hair conditioning mixture (step I). In order to maintain the desired cool tone of the colored hair, the pigmentation agent is incorporated into the hair conditioning mixture until homogenously combined, thereby forming a pigmented hair conditioning mixture (step J). Subsequently, a hair conditioning process is performed with the pigmented hair conditioning mixture, without losing the cool tone of the developed hair color (step K). It should also be noted that, based on the consistency, texture, and chemical state of the combining product, the pigmentation agent may be in a state of at least one of micro capsules, liquid, granules, and creme. This allows for easy combination and creation of a uniform homogenous mixture.

In the preferred embodiment, it is preferred that the pigmented hair bleaching mixture provides a cool toned blonde hair color. However, it should be note that the method of the present invention may be applicable towards achieving any hair color, hair style, and/or hair treatment processes, as long as the intents of the present invention are not altered.

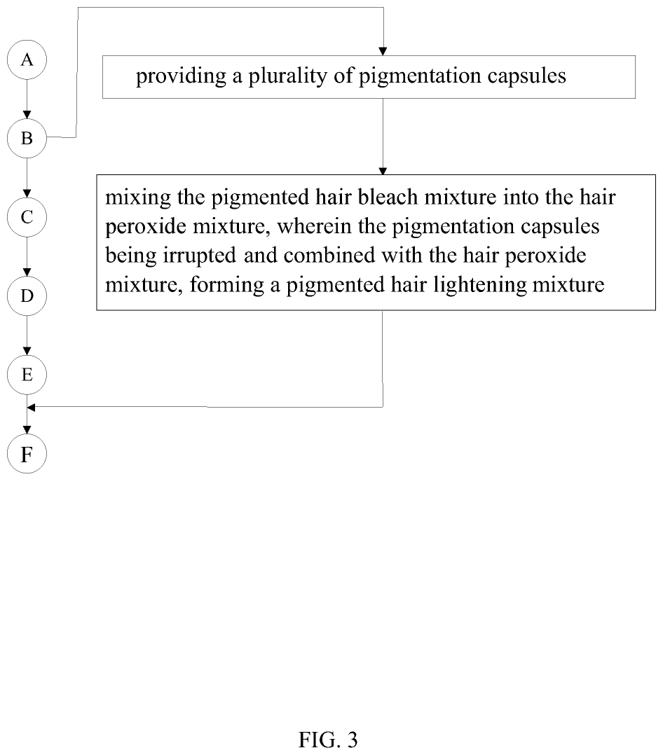

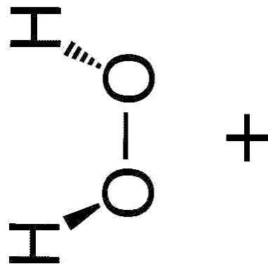

In the preferred embodiment of the method of the present invention, the pigmentation agent may comprise micro capsules. Accordingly, as seen in FIG. 3, a sub process of the overall method includes the following steps. The method begins by providing a plurality of pigmentation capsules as the pigmentation agent. Subsequently, as seen in FIG. 1, the plurality of pigmentation capsules is incorporated into the hair bleach mixture to form the pigmented hair bleach mixture. Further, the subprocess continues by mixing the pigmented hair bleach mixture with the hair peroxide mixture to form a pigmented hair lightening mixture, wherein the pigmentation capsules get irrupted and combined with the hair peroxide mixture during the mixing process. The pigmented capsules are micro capsules which irrupt when mixed with the previously described developer during the mixing process. However, the present invention is not limited to such configurations, and the pigmented capsules may comprise any internal structure which allows for the same result within the chemical process. Furthermore, it is preferred that the hair bleach mixture is in powder form, for the pigmentation agent to be microcapsules. Following this, a hair bleaching process is performed with the pigmented hair lightening mixture, wherein the pigmentation capsules provide a cool tone effect while simultaneously removing unwanted yellow and orange undertones to hair during the hair bleaching process.

It is important to note that the presence of the pigmented capsules is also considered within the scope of cream color and/or toners. Therefore, any and all modification and/or variation which result from the present invention are still considered to be within the scope of the present invention. It is also important to note that the pigmented capsules are not limited to the application of obtaining blonde hair, but rather any and all possible applications which are derived from the present invention. However, it is important to note that the ingredients and components from the present invention are specifically focused and tailored for providing the most effective and healthy blonde resulting color.

The aforementioned description is only intended to describe and disclose the preferred embodiment for the present invention. However, any alternate embodiment which is derived or developed from the present invention is still to be considered within the scope of the present invention.

Lastly, it is important to reiterate that the pigmented hair bleach and/or pigmented hair peroxide is not limited to pigmented capsules but rather any type of pigmentation agent such as liquid pigmentation agent, granular pigmentation agent and/or any type of pigmentation agent.

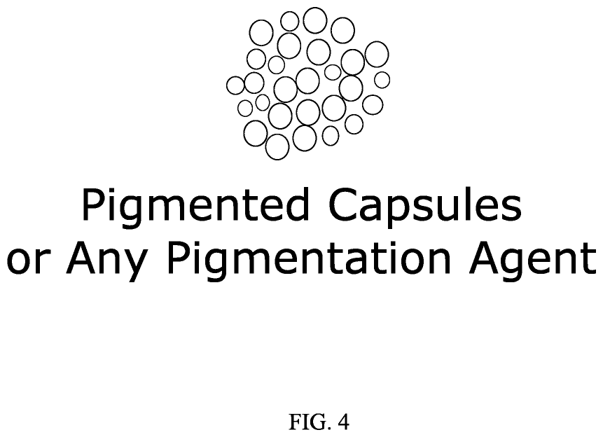

Additionally, FIGS. 4-7 are simply intended for the purpose of depicting and illustrating and the present invention. Therefore, the accompanying figures do not limit the scope of the application in any way shape or form.

The invention has been explained in relation to its preferred embodiment, but it is to be understood that many other possible modifications and variations can be made without departing from the spirit and scope of the invention. Obvious changes, modifications and substitutions may be made by those skilled in the art to achieve the same purpose of the invention. The exemplary embodiments are merely examples and are not intended to limit the scope of the invention. It is intended that the present invention cover all other embodiments that are within the scope of the appended claims and their equivalents.

Although the invention has been explained in relation to its preferred embodiment, it is to be understood that many other possible modifications and variations can be made without departing from the spirit and scope of the invention as hereinafter claimed.

What is claimed is:

1. A method of combining pigments in hair coloring and related products, the method comprising:
(A) providing a hair bleach mixture;
(B) providing a pigmentation agent;
(C) providing a hair peroxide mixture;
(D) incorporating the pigmentation agent into at least one of the hair bleach mixture and the hair peroxide mixture to form a pigmented mixture.
(E) mixing the hair bleach mixture with the hair peroxide mixture to form a pigmented hair lightening mixture; and
(F) performing a hair bleaching process with the pigmented hair lightening mixture, wherein the pigmentation agent provides a cool tone effect while simultaneously removing unwanted yellow and orange undertones to hair during the hair bleaching process.

2. The method of claim 1, wherein the pigmented hair lightening mixture providing blonde hair color.

3. The method claim 1, wherein the hair peroxide mixture comprising vegetable collagen.

4. The method of claim 1, further comprising:
(G) providing a plurality of hair care and hair styling mixtures;
(H) incorporating the pigmentation agent to at least one of the plurality of hair care and hair styling mixtures.

5. The method of claim 1, further comprising:
(I) providing a hair conditioning mixture;
(J) incorporating the pigmentation agent to the hair conditioning mixture until homogenously combined, forming a pigmented hair conditioning mixture;
(K) performing a hair conditioning process with the pigmented hair conditioning mixture.

6. The method of claim 1, wherein the pigmentation agent being in a state of at least one of micro capsules, liquid, granules, and creme.

7. The method of claim 1, wherein the pigmented hair bleach mixture and pigmented hair peroxide mixture providing a cool toned blonde hair color.

8. A method of combining pigments in hair dyeing and related products, the method comprising:
providing a hair bleach mixture;
providing a plurality of pigmentation capsules;
providing a hair peroxide mixture;
incorporating the plurality of pigmentation capsules into the hair bleach mixture to form a pigmented hair bleach mixture;
mixing the pigmented hair bleach mixture with the hair peroxide mixture to form a pigmented hair lightening mixture, wherein the pigmentation capsules being irrupted and combined with the hair peroxide mixture during the mixing process;
performing a hair bleaching process with the pigmented hair lightening mixture, wherein the pigmentation capsules provide a cool tone effect while simultaneously removing unwanted yellow and orange undertones to hair during the hair bleaching process.

9. The method of claim 8, wherein the pigmented hair lightening mixture providing a cool toned blonde hair color.

10. The method claim 8, wherein the hair peroxide mixture comprising vegetable collagen.

11. The method of claim 8, wherein the hair bleach mixture being in powder form.

12. A method for providing desired color tone in hair coloring process, the method comprising:
providing a hair bleach mixture;
providing a pigmentation agent;
providing a hair peroxide mixture;
providing a hair conditioning mixture;
incorporating the pigmentation agent into the hair bleach mixture to form a pigmented hair bleach mixture;
incorporating the pigmentation agent into the hair peroxide mixture to form a pigmented hair peroxide mixture;
mixing the pigmented hair bleach mixture with the pigmented hair peroxide mixture to form a pigmented hair lightening mixture;
performing a hair bleaching process with the pigmented hair lightening mixture, wherein the pigmentation agent provides a cool tone effect while simultaneously removing unwanted yellow and orange undertones to hair during the hair bleaching process;
incorporating the pigmentation agent into the hair conditioning mixture until well combined to form a pigmented hair conditioning mixture; and
performing a conditioning process with the pigmented hair conditioning mixture.

13. The method of claim 12, wherein the pigmented hair lightening mixture providing blonde hair color.

14. The method of claim 12, wherein the hair peroxide mixture comprising vegetable collagen.

15. The method of claim 12, comprising:
providing a plurality of hair coloring and hair styling mixtures;
incorporating the pigmentation agent to at least one of the plurality of hair coloring and hair styling mixtures.

16. The method of claim 12, wherein the pigmentation agent being in a state of at least one of micro capsules, liquid, granules, and creme.

17. The method of claim 12, wherein the pigmented hair bleach mixture and pigmented hair peroxide mixture providing a cool toned hair color.
